(19) United States
(12) Patent Application Publication
(10) Pub. No.: US 2023/0201516 A1
(43) Pub. Date: Jun. 29, 2023

(54) BREATH DETECTION APPARATUS AND METHOD FOR BREATH DETECTION

(71) Applicant: PEMDX PTY LTD, West End, QLD (AU)

(72) Inventors: Phillip MELKSHAM, Red Hill, Queensland (AU); Dylan Ray ROWE, Red Hill, Queensland (AU); Mariam ROWE, Red Hill, Queensland (AU)

(73) Assignee: PEMDX PTY LTD, West End, QLD (AU)

(21) Appl. No.: 18/000,337

(22) PCT Filed: Jun. 17, 2021
(86) PCT No.: PCT/AU2021/050626

§ 371 Date: Nov. 30, 2022

(30) Foreign Application Priority Data
Jun. 17, 2020 (AU) 2020902011

(51) Int. Cl. A61M 16/16

(52) U.S. Cl. CPC A61M 16/161; A61M 2205/3368

(57) ABSTRACT

A breath detection apparatus for monitoring individual breaths of a patient. The apparatus includes a sensing assembly comprising a humidity sensor. The sensing assembly adapted to be connected to an oxygenation device wherein respiratory gases of a patient are directed over the sensing assembly and the humidity sensor is adapted to monitor differences in humidity during a breath of the patient. The humidity sensor generates an electrical signal representing a humidity level in response to measuring the humidity during the breath. The apparatus also includes a processor in electrical communication with the sensing assembly to receive electrical signals generated by the humidity sensor and determine a state of the relative humidity during the breath, and a visual display element controlled by the processor and adapted to display a predetermined visual alert in response to the state of the relative humidity during the breath.

### FIELD

The present invention relates to a breath detection apparatus and method for breath detection.

### BACKGROUND

Any references to methods, apparatus or documents of the prior art are not to be taken as constituting any evidence or admission that they formed, or form part of the common general knowledge.

The monitoring of human respiration in conscious, sedated and unconscious patients has long been a perplexing problem that has, in some ways, been overcome by advances in technology.

The medical profession has devised a number of techniques to monitor and determine respiration and respiratory qualities of a patient. Some of these techniques include physically observing chest excursion or chest auscultation, misting on a mirror and movement of air flow from the nasal passages. However, these techniques can be prone to human error and may produce unreliable results.

Some more advanced monitoring techniques include measuring the amount of carbon dioxide (CO₂) expired from respiratory gases. The measurement of carbon dioxide in expired respiratory gases is a known method for use with intubated patients (i.e. patients having a tube placed into the trachea).

However, during the perioperative period, the monitoring of CO₂ in respiratory gases from patients is often not undertaken, typically for logistical and resource related reasons. Instead, during this time, the respiratory monitoring and breath detection is conducted by the less reliable practices of observation and examination described above.

### OBJECT

It is an aim of this invention to provide a breath detection apparatus and/or a method for breath detection which overcomes or ameliorates one or more of the disadvantages or problems described above, or which at least provides a useful commercial alternative.

Other preferred objects of the present invention will become apparent from the following description.

### SUMMARY OF THE INVENTION

According to a first aspect of the present invention there is provided a breath detection apparatus for monitoring individual breaths of a patient, the apparatus comprising:

a sensing assembly comprising a humidity sensor, the sensing assembly adapted to be connected to an oxygenation device wherein respiratory gases of a patient are directed over the sensing assembly, the humidity sensor adapted to monitor differences in humidity during a breath of the patient, wherein the humidity sensor generates an electrical signal representing a humidity level in response to measuring the humidity during a breath;

a processor in electrical communication with the sensing assembly to receive electrical signals generated by the humidity sensor and determine a state of the relative humidity during a breath; and

a visual display element controlled by the processor and adapted to display a predetermined visual alert in response to the state of the relative humidity during a breath.

Preferably, the apparatus further comprises a housing adapted to connect to an oxygenation device, the housing having the sensing assembly located therein and adapted to be in fluid communication with the oxygenation device to receive respiratory gases exhaled from the patient to flow over the sensing assembly.

Preferably, the sensing assembly is adapted to monitor differences in humidity during an exhalation and/or an inhalation of a respiratory cycle.

Preferably, the sensing assembly comprises only a humidity sensor. Alternatively, the sensing assembly comprises a humidity sensor and a temperature sensor. Preferably, the sensing assembly comprises only a humidity sensor and a temperature sensor.

According to a second aspect of the present invention there is provided a breath detection apparatus for monitoring individual breaths of a patient, the apparatus comprising:
a housing adapted to connect to an oxygenation device, the housing having a substantially enclosed cavity adapted to be in fluid communication with the oxygenation device to receive respiratory gases exhaled from the patient into the cavity; and
a sensing assembly located within the substantially enclosed cavity adapted to monitor the cavity during a breath, the sensing assembly comprising:
   a humidity sensor adapted to monitor humidity in the cavity (or generally monitor the humidity of the respiratory gases) during a breath, wherein the humidity sensor generates a first electrical signal representing a humidity level in response to measuring the humidity of the cavity;
   a pressure sensor adapted to monitor differences in pressure in the cavity during a breath, wherein the pressure sensor generates a second electrical signal representing a pressure level in response to measuring the humidity of the cavity; and
   a temperature sensor adapted to monitor differences in temperature in the cavity during a breath, wherein the temperature sensor generates a third electrical signal representing a temperature level in response to measuring the humidity of the cavity;
a processor in electrical communication with the sensing assembly to receive the first, second and third electrical signals generated by the humidity sensor, pressure sensor and temperature sensor and determine a breathing state during a breath; and
a visual display element controlled by the processor and adapted to display a predetermined visual alert in response to the breathing state during a breath

Preferably, the oxygenation device comprises a mask element. Preferably, the mask element comprises a nose and mouth covering portion.

Preferably, the oxygenation device comprises an airway catheter device comprising an endotracheal tube (ETT) or laryngeal mask airway (LMA). Preferably, the oxygenation device comprises high flow nasal oxygen prongs or an oral mouth guard.

Preferably, the housing comprises an adapter element adapted to connect to the oxygenation device. More preferably, the adapter element is adapted to connect to an oxygen mask or airway catheter device.

Preferably, the visual display element comprises an electronically controlled light in electrical communication with the processor. Preferably, the processor is programmed to activate the electronically controlled light in response to the breathing state.

Preferably, the apparatus further comprises an audible alert element. Preferably, the audible alert element comprises an electronically controlled audible alarm connected to the processor.

In another aspect, the invention provides a breath detection apparatus comprising:

an oxygenation device adapted to assist a patient with breathing;

a housing connected to the oxygenation device, the housing having a substantially enclosed cavity that is in fluid communication with the oxygenation device to receive respiratory gases exhaled from the patient into the cavity; and

a sensing assembly comprising a humidity sensor located within the substantially enclosed cavity, the humidity sensor adapted to monitor differences in humidity in the cavity (or generally monitor the humidity of the respiratory gases) during a breath, wherein the humidity sensor generates an electrical signal representing a humidity level in response to measuring the humidity of the cavity;

a processor in electrical communication with the sensing assembly to receive electrical signals generated by the humidity sensor and determine a state of the relative humidity of the cavity during a breath; and

a visual display element controlled by the processor and adapted to display a predetermined visual alert in response to the state of the relative humidity of the cavity during a breath.

In another aspect, the invention resides in a method for breath detection, the method comprising the steps of:

electronically monitoring a humidity level by a humidity sensor connected to an oxygenation device connected to a patient, wherein the humidity sensor receives respiratory gases exhaled from the face and wherein the humidity sensor generates an electrical signal representing a humidity value in response to measuring the humidity during a breath;

processing the electrical signal to determine a state of the relative humidity during a breath; and

generating a predetermined visual alert in response to the state of the relative humidity during a breath.

Preferably, the step of electronic monitoring the humidity level comprises measuring humidity at a first time during a breath and a second time during the breath. Preferably, the humidity is measured greater than 3 times a second. Preferably, the humidity is measured every 250 ms.

In another aspect, the invention resides in a method for respiratory monitoring, the method comprising the steps of:

electronically monitoring a humidity level by a humidity sensor of a substantially enclosed cavity of the housing connected to an assistive breathing device connected to a patient, wherein the cavity or sensing assembly receives respiratory gases exhaled from the face and wherein the humidity sensor generates an electrical signal representing a humidity value in response to measuring the humidity of the cavity;

processing the electrical signal to determine a state of the relative humidity of the cavity during a breath; and

generating a predetermined visual alert in response to the state of the relative humidity of the cavity during a breath.

Preferably, the method further comprises the step of measuring a first humidity level of the cavity at a first time; and

measuring a second humidity level of the cavity at a second time.

Preferably, the second time is after the first time. Preferably, the second time is no greater than 500 ms after the first time. Preferably, the second time is between 100 ms and 500 ms after the first time. Preferably, the second time is between 150 ms and 400 ms after the first time. Preferably, the second time is between 200 ms and 300 ms after the first time. Preferably, the second time is no greater than 250 ms after the first time.

Preferably, the method further comprises the step of converting the electronic signal to the humidity value by an Analog to Digital Converter. Preferably, the method further comprises the step of the first humidity level and the second humidity level to a corresponding value by an Analog to Digital Converter.

Preferably, the method further comprises the step of comparing the first humidity level with the second humidity level to determine the status of the relative humidity of the cavity during a breath. Preferably, the method further comprises the step of subtracting the first humidity level from the second humidity level.

Preferably, the state of the relative humidity of the cavity comprises one of the following:
a. a change in the relative humidity of the cavity during a breath; or
b. no change in the relative humidity of the cavity during a breath.

More preferably, the state of the relative humidity of the cavity comprises one of the following:
a. an increase in the relative humidity of the cavity during a breath; or
b. a decrease in the relative humidity of the cavity during a breath; or
c. no change in the relative humidity of the cavity during a breath.

Preferably, the predetermined visual alert is a unique visual alert based on the determined state of the relative humidity of the cavity during a breath.

In yet another aspect, the invention resides in a method for breath detection, the method comprising the steps of:

electronically monitoring a humidity level by a humidity sensor of a substantially enclosed cavity of an oxygenation device connected to a patient, wherein the cavity or sensing assembly receives respiratory gases exhaled from the patient and wherein the humidity sensor generates a first electrical signal representing the humidity level in response to measuring the humidity of the cavity;

electronically monitoring a pressure level by a pressure sensor of the cavity of the oxygenation device connected to the patient, wherein the pressure sensor generates a second electrical signal representing the pressure level in response to measuring the pressure of the cavity;

electronically monitoring a temperature level by a temperature sensor of the cavity of the oxygenation device connected to the patient, wherein the temperature sensor generates a third electrical signal representing the temperature level in response to measuring the temperature of the cavity;

processing each of the first, second and third electrical signals to determine a breathing state during a breath; and

generating a predetermined visual alert in response to the breathing state during a breath.

Preferably, the method further comprises the step of measuring:

a first humidity level of the cavity at a first time;

a first temperature level of the cavity at the first time;

a first pressure level of the cavity at the first time;

measuring a second humidity level of the cavity at a second time;

a second temperature level of the cavity at the second time; and

a second pressure level of the cavity at the second time.

Preferably, the method further comprises the step of measuring:

a first humidity level at a first time;

a first temperature level at the first time;

a first pressure level at the first time;

measuring a second humidity level at a second time;

a second temperature level at the second time; and

a second pressure level at the second time.

Preferably, the second time is after the first time.

Preferably, the method further comprises the step of converting each of the pressure, temperature and humidity levels to a corresponding value by an Analog to Digital Converter.

Preferably, the method further comprises the step of comparing each of the first humidity, pressure and temperature levels with the corresponding second humidity, pressure and temperature levels to determine the breathing state during a breath. Preferably, the method further comprises the step of subtracting the first humidity level from the second humidity level, subtracting the first pressure level from the second pressure level, and subtracting the first temperature level from the second temperature level. Preferably, the step of comparing each of the first humidity, pressure and temperature levels with the corresponding second humidity, pressure and temperature levels to determine the breathing state during a breath occurs two or more times a second. Preferably, the step of comparing each of the first humidity, pressure and temperature levels with the corresponding second humidity, pressure and temperature levels to determine the breathing state during a breath occurs three or more times a second. Preferably, the step of comparing each of the first humidity, pressure and temperature levels with the corresponding second humidity, pressure and temperature levels to determine the breathing state during a breath occurs every 250 ms.

Preferably, the breathing state comprises one of the following:
a. a change in each of the humidity, pressure and temperature in the cavity during a breath; or
b. no change in each of the humidity, pressure and temperature in the cavity during a breath.

More preferably, the breathing state comprises one of the following:
a. an increase in each of the humidity, pressure and temperature in the cavity during a breath; or
b. a decrease in each of the humidity, pressure and temperature in the cavity during a breath; or
c. no change in each of the humidity, pressure and temperature in the cavity during a breath.

Preferably, the predetermined visual alert is a unique visual alert based on the determined breathing state during a breath.

Preferably, the apparatus further comprises a wireless communication device for transmitting the predetermined visual alert to a display device. Preferably, the wireless communication device is connected to the processor. Preferably, the wireless communication device comprises at least one of a Wi-Fi transmitter, a Bluetooth transmitter, and a Near Field Communication (NFC) transmitter.

Preferably, the method further comprises wirelessly transmitting the predetermined visual alert to a display device.

In yet another aspect, the invention resides in a breath detection apparatus for monitoring individual breaths, the apparatus comprising:

a housing having a substantially enclosed cavity adapted to receive respiratory gases exhaled from a user into the cavity;

a sensing assembly comprising a humidity sensor located within the substantially enclosed cavity, the humidity sensor adapted to monitor differences in humidity in the cavity during a breath, wherein the humidity sensor generates an electrical signal representing a humidity level in response to measuring the humidity of the cavity;

a processor in electrical communication with the sensing assembly to receive electrical signals generated by the humidity sensor and determine a state of the relative humidity of the cavity during a breath; and

a visual display element controlled by the processor and adapted to display a predetermined visual alert in response to the state of the relative humidity of the cavity during a breath.

In yet another aspect, the invention resides in a method for breath detection, the method comprising the steps of:

electronically monitoring a humidity level by a humidity sensor of a substantially enclosed cavity of a housing located adjacent a nose and/or mouth of a user, wherein the cavity receives respiratory gases exhaled from the user and wherein the humidity sensor generates an electrical signal representing a humidity value in response to measuring the humidity of the cavity;

processing the electrical signal to determine a state of the relative humidity of the cavity during a breath; and

generating a predetermined visual alert in response to the state of the relative humidity of the cavity during a breath.

According to another aspect of the present invention there is provided a breath detection apparatus for monitoring individual breaths of a patient, the apparatus comprising:

a housing adapted to connect to an oxygenation device, the housing having a substantially enclosed cavity adapted to be in fluid communication with the oxygenation device to receive respiratory gases exhaled from the patient into the cavity;

a sensing assembly comprising a humidity sensor located within the substantially enclosed cavity, the humidity sensor adapted to monitor differences in humidity in the cavity during a breath, wherein the humidity sensor generates an electrical signal representing a humidity level in response to measuring the humidity of the cavity;

a processor in electrical communication with the sensing assembly to receive electrical signals generated by the humidity sensor and determine a state of the relative humidity of the cavity during a breath; and

a visual display element controlled by the processor and adapted to display a predetermined visual alert in response to the state of the relative humidity of the cavity during a breath.

In another embodiment, the present invention provides a breath detection system for monitoring individual breaths of a patient, the apparatus comprising:

an oxygen mask having a ventilation port;

a housing having an opening and a sensing assembly comprising a humidity sensor therein, the housing positioned over at least a portion of the ventilation port to direct respiratory gases of a patient wearing the oxygen mask into the opening of the housing and over the sensing assembly, the humidity sensor adapted to monitor differences in humidity during a breath of the patient, wherein the humidity sensor generates an electrical signal representing a humidity level in response to measuring the humidity during the breath;

a processor in electrical communication with the sensing assembly to receive electrical signals generated by the humidity sensor and determine a state of the relative humidity during the breath; and

a visual display element controlled by the processor and adapted to display a predetermined visual alert in response to the state of the relative humidity during the breath.

Further features and advantages of the present invention will become apparent from the following detailed description.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 illustrates a breath detection apparatus according to an embodiment of the present invention for monitoring individual breaths of a patient;

FIGS. 2(a) and 2(b) illustrate a printed circuit board (PCB) for monitoring the humidity of the respiratory gases;

FIG. 3 illustrates a schematic of the PCB of the breath detection apparatus;

FIG. 4 illustrates a block diagram of the PCB of the breath detection apparatus;

FIG. 5 illustrates pseudo-code implemented on a microcontroller of the PCB to control the apparatus in accordance with an embodiment of the invention;

FIG. 6 illustrates a flow diagram of a method coded as instructions in digital memory and executed by a processor;

FIG. 7a illustrates an example of an irregular breathing pattern due to apnoea;

FIG. 7b illustrates an ideal data stream from breath detection;

FIG. 7c illustrates a realistic data stream from breath detection when using an “open system” that is not stability controlled;

FIG. 8 is an exemplary interface screen display for presenting results of the breath detection apparatus on a smart device (such as a smartphone or tablet);

FIG. 9 illustrates a breath detection apparatus according to an embodiment of the present invention for monitoring individual breaths of a user;

FIG. 10 illustrates a breath detection apparatus according to an embodiment of the present invention for monitoring individual breaths of a user; and

FIG. 11 illustrates an exploded view of the breath detection apparatus of FIG. 10.

### DETAILED DESCRIPTION OF THE PREFERRED EMBODIMENTS

Embodiments of the invention described herein relate to a real-time breath indicating and detecting apparatus with a very high accuracy, ideally greater than 98%. The apparatus is intended to provide a very low-cost, single-use, disposable breath indicator that does not need any ancillary equipment such as a monitor or external analysis machine. This is in contrast to existing systems which focus on measuring a time-series of data which can be analysed or displayed as a waveform graph to gain an assessment of respiratory rate, which is distinct from breath indication and detection.

The terms “breath” and “during a breath” are used to refer to a single breath or phase of a respiratory cycle (i.e. an individual exhalation or inhalation) unless otherwise indicated.

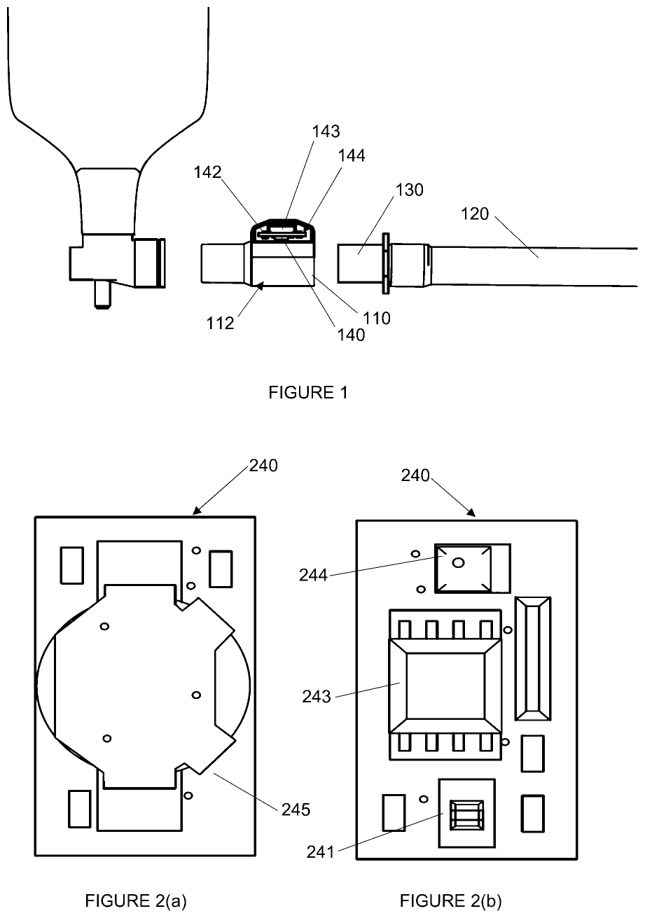

FIG. 1 illustrates a breath detection apparatus 100 according to a preferred embodiment of the present invention for monitoring individual breaths of a patient. The breath detection apparatus 100 comprises a housing 110. In the illustrated embodiment, the housing 110 is adapted to connect to an oxygenation device in the form of a laryngeal mask airway (LMA) 120 via an adapter 130. However, in some alternative embodiments, as described below, the housing may not be adapted to connect to an oxygenation device or any secondary device or apparatus.

While the embodiment of the apparatus shown is connected to an LMA, it will be appreciated that the apparatus can be used with a number of oxygenation devices, such as an oxygen face mask, airway catheter device (such as an endotracheal tube (ETT), high flow nasal oxygen cannula, oral mouth guard, for example).

The housing 110 includes a substantially enclosed cavity 112 that is in fluid communication with the LMA 120 via the adapter 130 to receive expired respiratory gases from a patient (not shown) connected to the LMA 120. Located within the enclosed cavity 112 of the housing 110 is a sensing system in the form of a printed circuit board (PCB) 140 configured to monitor the humidity, pressure (optionally) and temperature of the enclosed cavity 112 as respiratory gases move through the enclosed cavity 112 due to respiratory cycles (i.e. inhalation and exhalation) of the patient.

The substantially enclosed cavity 112 is substantially enclosed by a body of the housing 110. However, the substantially enclosed cavity 112 must allow fluids and in particular respiratory gases, to pass through and thus over the PCB 140 and sensing assembly allowing changes in humidity, temperature and pressure to be monitored in each individual breath (i.e. each individual inhalation or exhalation). The Inventors have found that the housing 110 does not require a substantially enclosed cavity to operate effectively. The Inventors have found that the apparatus 100 performs in a substantially open air environment and only requires that the apparatus be placed adjacent the nose and/or mouth of a patient.

The PCB 140 includes a sensing assembly comprising a humidity sensor 142 adapted to monitor differences in humidity during a breath in the substantially enclosed cavity 112 of the housing 110 during a breath. While the humidity sensor 142 is not orientated in any specific way, the Inventors have found that when placed proximal to a Heat and Moisture Exchange (HME) filter in a breathing circuit of a ETT or LMA, the likelihood of the humidity approaching 100% and adversely affecting the ability of the humidity sensor to reliably monitor respiration is reduced.

The PCB 140 also includes a computing device in the form of microcontroller 143 and a light emitting diode (LED) 144 for visually displaying a state of the relative humidity of the breath during a breath as the conditions of the enclosed cavity 112 are affected by the respiration (or lack thereof) of the patient connected to the LMA 120. The term “respiration” as used herein should be taken to refer to breath detection or breath indication.

The humidity sensor 142 electronically monitors a humidity level of the enclosed cavity 112 of the housing 110 which is in fluid communication with the LMA 120 connected to a patient. The enclosed cavity 112 receives respiratory gases exhaled by the patient and, in response, the humidity sensor generates an electrical signal representing a humidity value in response to measuring the humidity of the enclosed cavity 112. The electrical signal representing the humidity value is communicated to a processor of the microcontroller 143 which computationally processes the electrical signal to determine a state of the relative humidity of the enclosed cavity 112 during a breath. This is achieved by sampling the humidity at different points in time and comparing the respective values. In a preferred embodiment, after every pair of breaths, the humidity in each breath is compared.

The microcontroller 143 then generates a predetermined visual alert in response to the determine state of the relative humidity of the enclosed cavity 112 during a breath. In particular, the processor of the microcontroller 143 sends an electrical signal to the LED 144 to display a specific colour that has been predetermined (and pre-programmed) to be representative of the state of the enclosed cavity 112. In one example, where no change in the humidity has been detected for at least 10 seconds, medically this indicates that the patient may not be breathing and the processor has been programmed to cause the LED to flash red to indicate a problem to observing medical staff.

Turning to FIGS. 2-4, another embodiment of a PCB 240 suitable for use with housing 110 described above. Similar to PCB 140, the PCB 240 includes the aforementioned humidity sensor located in an environmental sensor 241 (a BME280 environmental sensor), a light in the form of a Red-Green-Blue (RGB) light emitting diode (LED) 244 and a computing device in the form of a microcontroller 243 (an Atmel low power 8-bit ATTiny85, in the illustrated embodiment, for example) that communicates with and controls the environmental sensor 243 and the RGB LED 244, which will be described in more detail below. The PCB 240 also includes a battery 245 (in the form of a 3 volt lithium CR1025 battery) located on the reverse side of the PCB 240 relative to the microcontroller 243.

The environmental sensor 241 includes a humidity sensor 241a (in the form of a capacitive sensor) for monitoring the relative humidity of the respiratory gases of the patient within the enclosed cavity of the housing, a temperature sensor 241b (in the form of a diode) and a pressure sensor 241c (in the form of a resistive sensor).

In some further embodiments, the environmental sensor only includes the humidity sensor or may have the pressure sensor and/or temperature sensor disabled or programmed to be skipped.

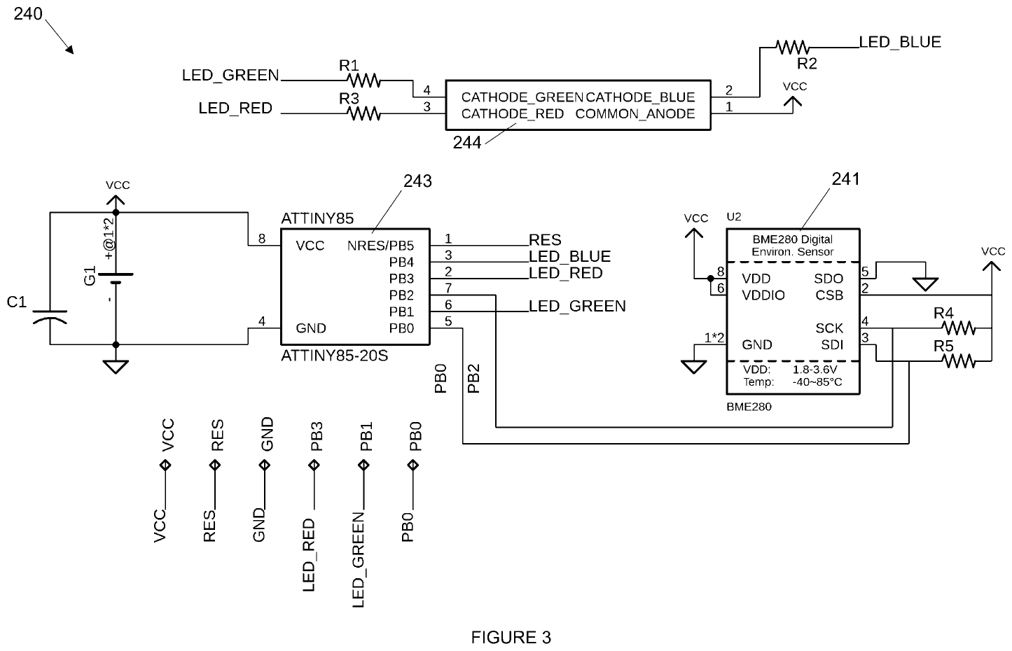

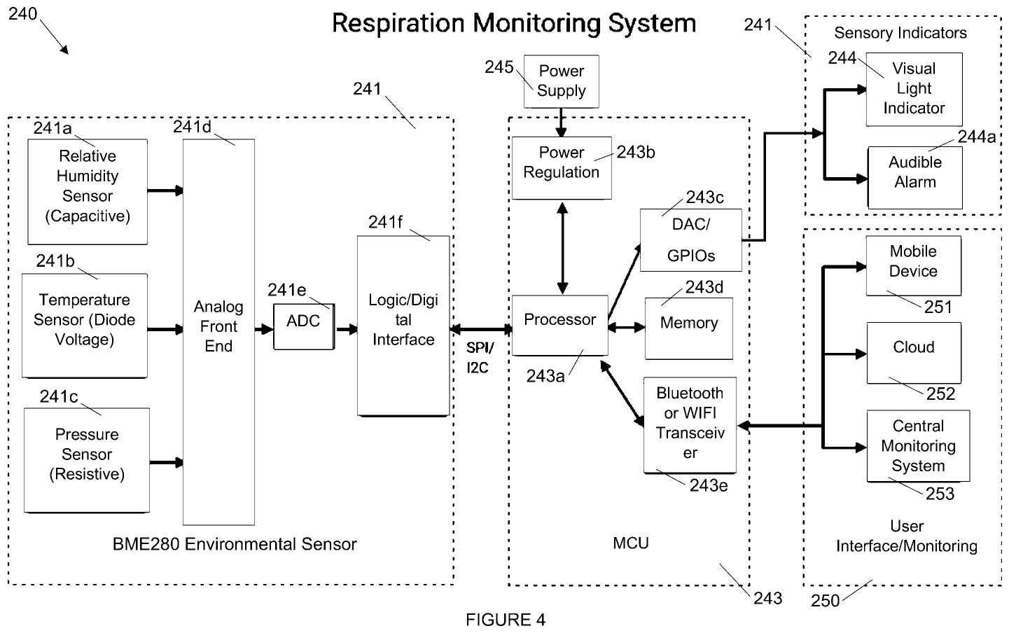

An exemplary schematic and block diagram of the PCB 240 can be seen in FIGS. 3 and 4. As shown in the diagrams, the environmental sensor 241 takes analog signal readings from the humidity sensor 241a, the temperature sensor 241b and the pressure sensor 241c, respectively, which are transmitted to the Analog Front End module 241d converted by the Analog-Digital Converter (ADC) 241e and passed to the digital interface 241f of the environmental sensor 241. With reference to the PCB schematic of FIG. 3, the SDI (data) and SCK (serial clock) pins are shown to output the data from the environmental sensor 241 to provide inputs to pins 5 (PB0) and 7 (PB2) of the microcontroller 243.

The environmental sensor 241 communicates with the processor 243a of the microcontroller 243 via the digital interface using a Serial Peripheral Interface (SPI) or Inter-integrated Circuit (I²C) protocol.

The microcontroller 243 further includes a power regulator 243b connected to a power supply (i.e. the battery 245), a Digital-Analog Converter (DAC) 243c, digital memory 243d and a Bluetooth/Wi-Fi transceiver 243e. In some embodiments, the PCB 240 further includes a Near-Field Communication (NFC) transmitter for wireless communication.

As shown, the processor 241a of the microcontroller 243 is in electrical communication with each of the power regulator 243b, DAC 243c, memory 243d and Bluetooth/Wi-Fi Transceiver 243e.

The processor 243a, via the DAC 243c, controls the RGB LED 244 and audible alarm 244a of the PCB 240 to visually, and in some embodiments audibly, indicate the condition of the patient being monitored.

The microcontroller 243 also communicates with a monitoring system 250 in the forms of a mobile device 251, cloud database 252 or central monitoring system 253 (such as a PC), in some embodiments, via the Bluetooth/Wi-Fi Transceiver 243e.

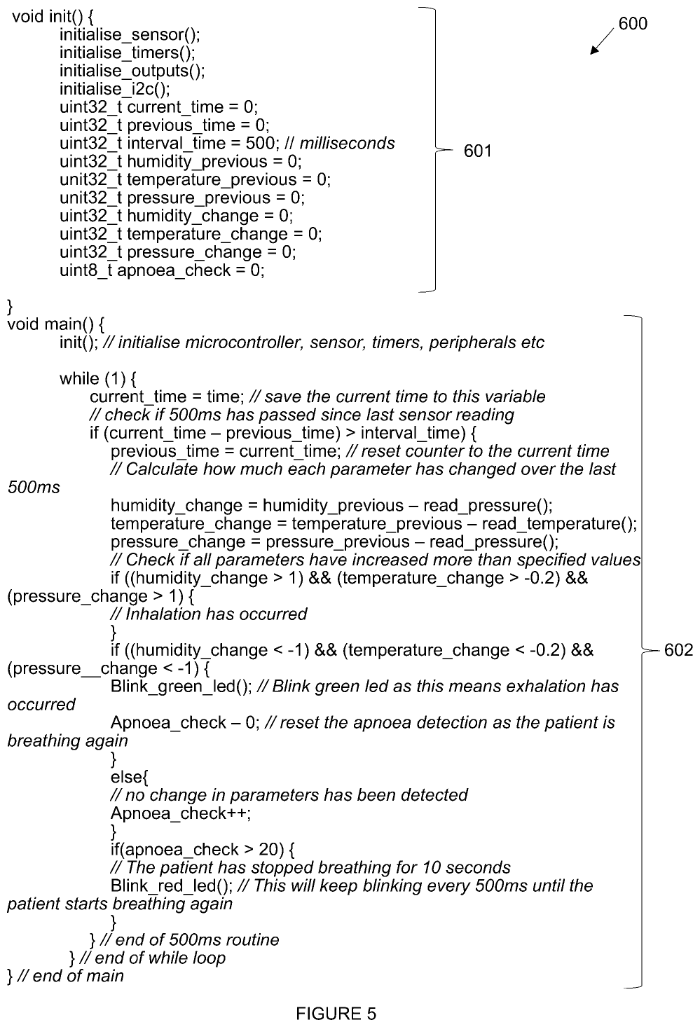

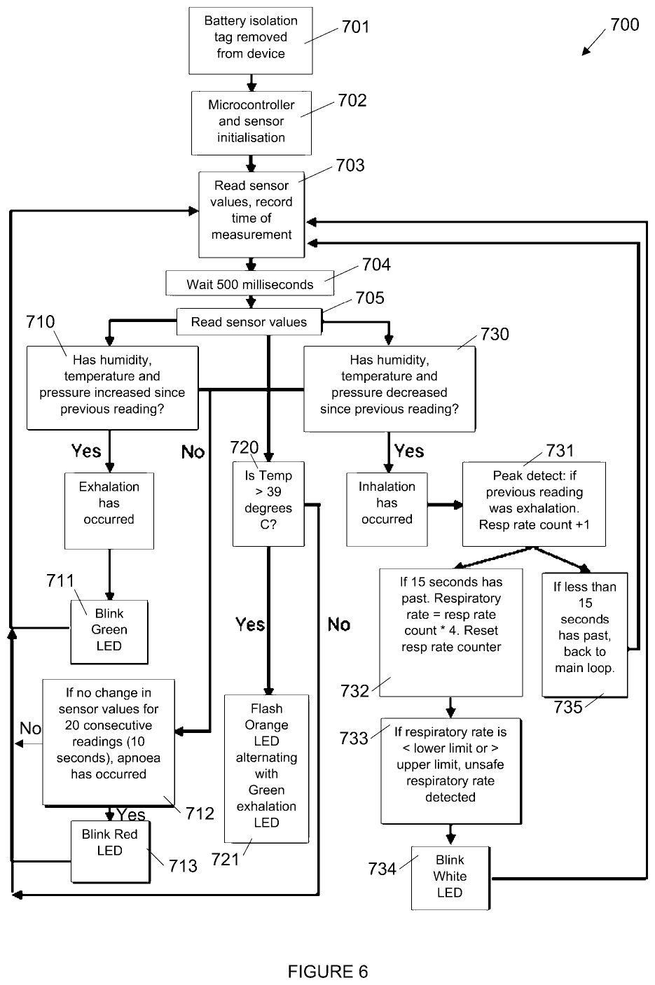

Examples of the operation of the PCB 240 are provided in the pseudo-code of FIG. 5 and flow diagram of FIG. 6.

With reference to FIG. 5, the pseudocode 600 implementable in software to control PCB 240 of the breath detection apparatus is programmed to initialise the microcontroller 243, sensor assembly 241, timers and peripherals in function 601. As can be seen, many of the parameters (temperature, pressure, humidity) are initially set to 0. The exception being the interval_time parameter being set at 500 ms which sets the time between sensor readings.

After initialisation, the pseudocode 600, when implemented, causes the processor to enter a loop (main function 602) which continuously updates the current_time variable by reading the system time and then repeatedly checks for whether the time that has elapsed is greater than the interval_time variable (set at 500 ms in the present embodiment) by substracting the value of the previous_time variable (which is set based on the previous time a sensor reading occurred) from the value of the current_time variable and comparing the remainder with the value of the interval_time.

If the predefined value for intervening time has not elapsed, the program continues to update the current_time variable and conducting the comparison explained above.

If the predefined value for intervening time has elapsed, the program replaces the current value of the previous_time variable with the value of the current_time variable to reset the counter.

The program then calculates the change in humidity (humidity_change), temperature (temperature_change) and pressure (pressure_change) by reading values from each of the humidity sensor, temperature sensor and pressure sensor of the environmental sensor and subtracting those values from the previous values of the respective humidity, temperature and pressure variables (i.e. humidity_previous, temperature_previous, pressure_previous).

The processor then checks for a number of different events.

Firstly, the program checks if the each of the parameters has increased more than predetermined values. In the illustrated embodiment, the processor determines whether humidity_change>1, temperature_change>0.2 and pressure-change>1 which, if true, indicates an inhalation has occurred and no further routines are executed or required for such an event.

The program then monitors for exhalation by determining whether humidity_change<−1 to −0.5, temperature_change<−0.2 to −0.04 (and preferably<−0.12) and pressure-change<−3 to −1 (and preferably<−2) which, if true, indicates an exhalation has occurred. The program then executes the blink_green_led() subroutine which sends a signal to the LED 244 to cause it to flash green. The satisfaction of the criteria for exhalation also causes the processor to execute instructions to set the apnoea_check value to 0 as the patient is indicated to being breathing regularly.

In a particularly preferred embodiment, the program monitors for exhalation by determining whether humidity_change<−0.7, temperature_change<−0.12 and pressure-change<−2) which, if true, indicates an exhalation has occurred. In response, the program executes the blink_green_led() subroutine which sends a signal to the LED 244 to cause it to flash green. As noted above, the satisfaction of the criteria for exhalation also causes the processor to execute instructions to set the apnoea_check value to 0 as the patient is indicated to being breathing regularly.

If exhalation does not occur, then the program automatically increments the apnoea_check variable by 1.

Subsequently, the processor 243a checks whether the value of the apnoea_check value exceeds 20 which is indicative of no complete breathing cycle (inhalation and exhalation) for at least 10 seconds. If true, the program then executes the blink_red_led() subroutine which sends a signal to the LED 244 to cause it to flash or blink red at intervals of 500 ms until the patient is detected to be breathing regularly once again by the program registering that the conditions described above for inhalation and exhalation are satisfied.

The above program runs continuously to monitor the patients breathing status and indicate the current status and/or any changes via the LED 244.

In another embodiment, a breath detection apparatus in accordance with an embodiment of the invention as described herein is configured to execute method 700 to determine respiration, respiration rate, Apnoea and temperature and generate warnings accordingly.

At Step 701, a battery isolation tag is removed from the device to allow the battery to power the PCB.

At Step 702, the microcontroller and sensors of the environmental sensor are initialised in response to the power received from the battery.

At Step 703, the pressure, humidity and temperature sensors of the environmental sensor generate respective signals which are transmitted, via the ADC described above in FIG. 2, to the processor of the microcontroller which reads the sensor values and records the time of measurement. In a preferable embodiment, humidity and temperature sensors sample the humidity and temperature of the air surrounding the sensors to initialise the system.

At Step 704, the processor is programmed to execute wait or delay of approximately 500 ms before, at Step 705, the pressure, humidity and temperature sensors of the environmental sensor generate respective signals which are transmitted to the processor of the microcontroller which reads the current sensor values and records the time of measurement of the current values. The first reading from Step 703 then becomes the previous values.

The above step utilises a wait or delay of approximately 500 ms. However, the wait or delay can be anywhere between 100 ms and 500 ms, 150 ms and 450 ms and, preferably, between 250 ms and 350 ms. In a particularly preferred embodiment, the wait or delay is approximately 250 ms.

The method then enters one of Steps 710, 720 or 720 depending on the values of the sensors.

At Step 710, the processor determines whether each of the humidity, temperature and pressure have all increased by comparing the current values with the previous values. In a preferred embodiment, the comparison occurs after every pair of breaths from a patient.

Preferably, the processor subtracts the previous values (i.e. first measurement) from the current values (i.e. second measurement) to obtain the gradient difference. If the gradient difference between the 2 measurements is greater than a predetermined threshold then this is an exhale.

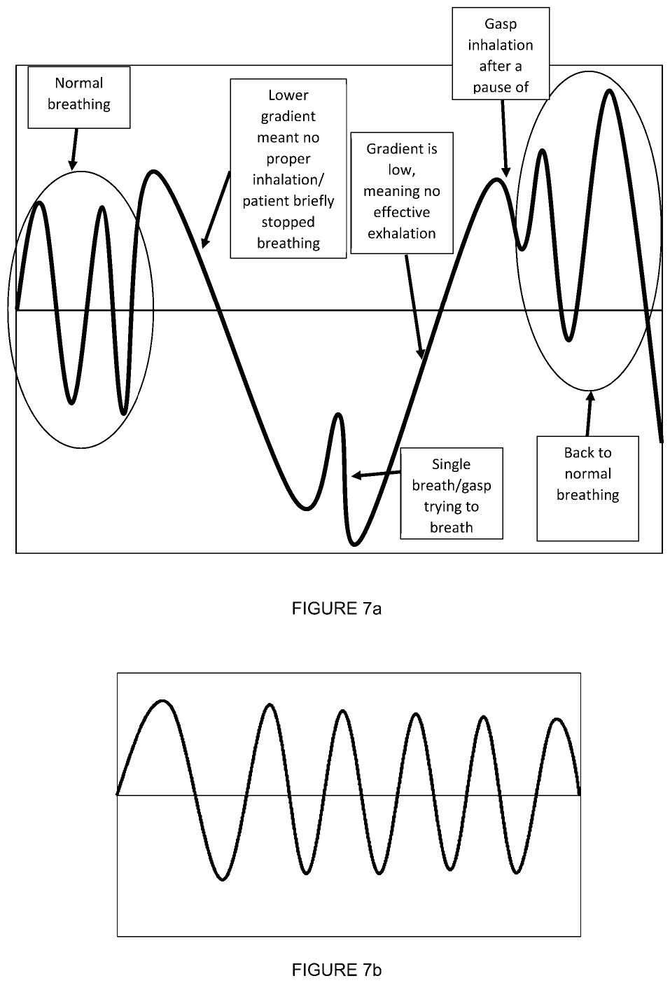

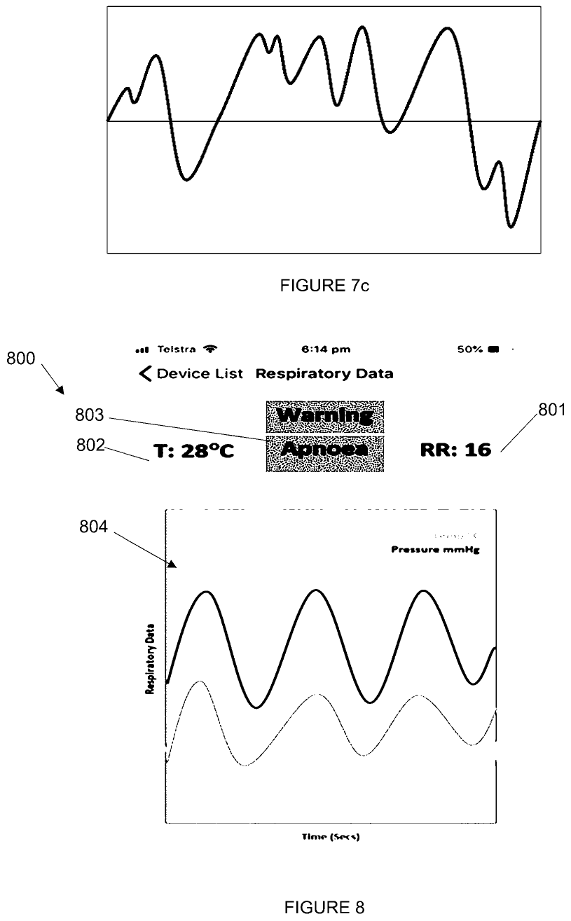

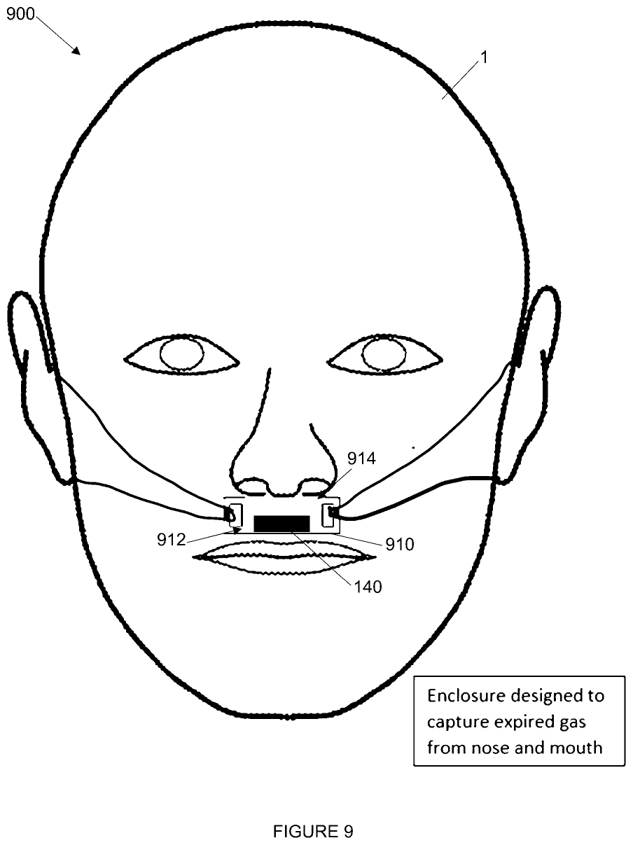

To determine the breathing state, the Inventors use inflection points of the exhaled gases. Thus, as the humidity measurement rapidly increases over the span of the delay, this would indicate an exhalation. By measuring over such a short time frame, the Inventors believe they have eliminated errors from drift, noise from inflowing oxygen, and false positives from other physiological effects not related to breathing. This also removes the need to have a stability-controlled sensor as the baseline for our measurement is completely irrelevant. An example is shown in FIG. 7a.

By using this very short time frame to determine exhalations, the Inventors believe the disclosed embodiment offers a number of advantages.

In particular, existing methods and systems maintain the sensors at a stable baseline temperature/humidity/pressure/flow, and then monitor a change from that baseline to determine respiratory rate. Embodiments of the present invention can use a single sensor which measures both temperature and humidity, sampling at greater than 3 times per second. A breath is determined based on a gradient change between each consecutive measurement. This means embodiments of the present invention do not need to keep the sensor stable, drift from environmental influences do not affect the monitor, and the device can be very small and made at a low-cost with no additional equipment required.

Existing systems often need to stabilise their sensor as the environment the device is measuring in is constantly changing due to physiological and environment changes. With reference to FIGS. 7b and 7c, there is shown an example expected temperature and humidity data during respiration versus the reality. In particular, FIG. 7c shows the data collected when using an open system that is not stability controlled (e.g. when only using a single humidity or temperature sensor near a person's face).

In a further advantage of embodiments of the present invention, the sensor is not required to be completely enclosed in a housing with the exhaled and inhaled gases.

Nevertheless, in some embodiments utilising a substantially enclosed sensor, the advantage of being completely enclosed is that the measurements will cycle around a fairly stable baseline meaning you could look at long time frames of data.

If true (in accordance with the parameters defined above), this indicates that an exhalation has occurred and the LED blinks green at Step 711.

If false, at Step 712, the processor checks if no change in sensor values has been recorded for more than 20 consecutive readings (i.e. 10 seconds) which indicates that the patient is suffering apnoea.

This executes Step 713 which causes the LED to blink red. The method then returns to Step 703.

If the sensor values have remained the same for 20 consecutive readings or less, the method returns to Step 703.

At Step 720, the method includes the step of detecting whether the temperate value of the temperature sensor exceeds 39° C. A temperature of greater than 39° C. is known to be a reliable indicator of the presence of an elevated airway temperature, which may indicate the occurrence of a respiratory issue.

If true, the method proceeds to Step 721 whereby an LED alternates between green and orange to indicate that breathing is normal but attention may be required.

If false, the method returns to Step 703.

At Step 730, the method includes the step of determining whether each of the humidity, temperature and pressure have all decreased by comparing the current values with the previous values.

If false, the method proceeds to Step 712 described above.

Alternatively, if true, this indicates inhalation has occurred, and the method proceeds to Step 731.

At Step 731, the method checks whether the previous reading indicated an exhalation. If true, a respiration counter (Respiration Count) is incremented by 1 in the memory of the microcontroller.

The method then checks the time that has elapsed since the exhalation.

If the time since the exhalation is 15 seconds or more, a calculation is performed at Step 732 as follows: Respiratory Rate=Respiration Count*4. The respiration counter is also reset to 0.

At Step 733, the method conducts a check to determine if the calculated Respiratory Rate is less than a lower limit (e.g. 6 breaths per minute) or greater than an upper limit (e.g. 20 breaths per minute). If true, this is known to be indicative of an unsafe respiratory rate and the method proceeds to Step 734 wherein the LED flashes or blinks a white colour.

If false, no unsafe respiratory rate is detected and the method returns to Step 703.

As indicated by Step 735, if the time since the exhalation is less than 15 seconds, no unsafe conditions have yet been detected and the method returns to Step 703.

In some further embodiments of the invention having an NFC transmitter, the breath detection apparatus is able to log historical respiratory rates and warnings that can be accessed at a single point for reference by medical staff.

In use, a breath detection apparatus as described herein but also including the NFC transmitter, is fitted to an oxygen face mask (or suitable oxygen mask) and turned on.

Embodiments of the present invention use humidity as a primary measurement with temperature as a secondary measurement.

The breath detection apparatus monitoring the expired gas of the patient and continuously logs the respiratory rate of the patient along with any alerts or warnings that are generated.

During a visit or check up of the patient by a medical staff member, the medical staff member can utilise an NFC reader (such as a capably equipped smartphone having an application for receiving and displaying the transmitted data).

In one particular advantage, the addition of the NFC transmitter can significantly improve the ability of medical staff to monitor and treat patients. An example of this can be seen in that if a patient stops breathing for minute on an ongoing basis (e.g. every 10 minutes), the attending medical staff member will be able to identify this apnoea from the historical data retrieved from the NFC transmitter whereas they would usually not be aware of such occurrences unless they happened to be present during an episode of apnoea.

Some exemplary embodiments for using the breath detection apparatus are described below.

In an embodiment of the breath detection apparatus in accordance with the invention as described herein used with an oxygenation device in the form of an ETT or LMA, the apparatus will be connected to the breathing circuit of the oxygenation device. The patient connected to the oxygenation device may be spontaneously breathing or mechanically ventilated.

An LED of the apparatus is illuminated green on expiration during respiration cycle.

If it is detected that respiration/breathing has ceased (as indicated by no changes in pressure, humidity and temperature within the breathing circuit) and respiration ceases for more than 10 seconds (which is the medical definition of the patient suffering apnoea), the LED will intermittently flash red. An audible buzzer or alarm may also be activated to indicate a dangerous breathing status of the patient.

In the event that respiration recommences, the LED will be illuminated green upon expiration and the red LED will no longer be active. In response to the alarm and/or visual alert, the doctor or attending medical staff can then assess the ventilation and make any corrections necessary for safe management of the patient.

If pressure within the breathing circuit is measured by the pressure sensor to be greater than 40 cmH₂O, the red LED and green LED alternatingly flash to indicate continued expiration by the patient but also an unsafe breathing condition. The audible alarm may also be activated.

If pressure is subsequently measured to be less than 40 cmH₂O, the red LED ceases flashing.

If temperature within the breathing circuit is measured by the temperature sensor to be greater than 39° C., the red LED and green LED alternatingly flash to indicate continued expiration by the patient but also an unsafe breathing condition.

If temperature is subsequently measured to be less than 39° C., the red LED ceases flashing.

In some embodiments, an application is available for smartphone devices (such as devices running the Android and iOS operating systems, for example) to graphically display both digital and analogue outputs of the measured parameters. Such an application will display alarms both visual and audible for apnoea, elevated pressure over 40 cmH₂O and temperature greater than 39° C. An example of such a graphical display 800 is provided in FIG. 7.

The values and warnings transmitted and displayed on graphical display 800 include respiratory rate 801 (as a value), respiratory temperature 802 (as a value), apnoea warnings 803. Respiratory data (including pressure, temperature and respiratory rate) is also displayed in a graph 804 to monitor changes in conditions during a breath.

The graphical display may also include temperature warnings and respiratory rate warnings in some embodiments.

Currently patients are transferred from the operating theatre (OT) to the Post Anaesthetic Care Unit (PACU) with LMAs in-situ while spontaneously breathing. Commonly the only monitoring used to detect respiration is observation and examination by a doctor. As the breath detection apparatus is able to remain attached to the patients LMA during transfer it allows for additional monitoring of respiration by the doctor. Once in the PACU, monitoring can continue and aid medical staff in recovery of the patient by ensuring apnoea's can be recognised and managed while the LMA is in place.

In other situations, monitoring of respiration can continue using a separate device attached to the oxygen face mask, once the patient has recovered, the LMA has been removed and a oxygen face mask placed on the patient for oxygen delivery. The device mounted on the oxygen face mask will again illuminated red and green LEDs as stated above in the breathing circuit. The Inventors have realised that pressure sensing may not be reliable because the breathing circuit is open. However, the breathing status of the patient can continue to be monitored using measurements of the humidity and temperature.

In some alternative embodiments, as shown in FIG. 8, there is provided a breath detection apparatus 900 comprising a housing 910 and a PCB 140 having a sensing assembly as described above.

The housing 910 includes a substantially enclosed cavity 912 having the PCB 140 located therein.

The housing 910 also includes an opening 914 to receive expired respiratory gases from a user 1 wearing the apparatus 900 into the substantially enclosed cavity 912.

As has been described herein, the PCB 140 is configured to monitor the humidity, pressure and temperature of the enclosed cavity 912 as respiratory gases move through the enclosed cavity 912 due to respiratory cycles (i.e. inhalation and exhalation) of the user 1.

The substantially enclosed cavity 912 is substantially enclosed by a body of the housing 910. However, the substantially enclosed cavity must allow fluids and in particular respiratory gases, to enter and thus pass over the PCB 140 and sensing assembly.

The breath detection apparatus 900 is envisioned to be particularly useful for detecting and monitoring obstructive sleep apnoea.

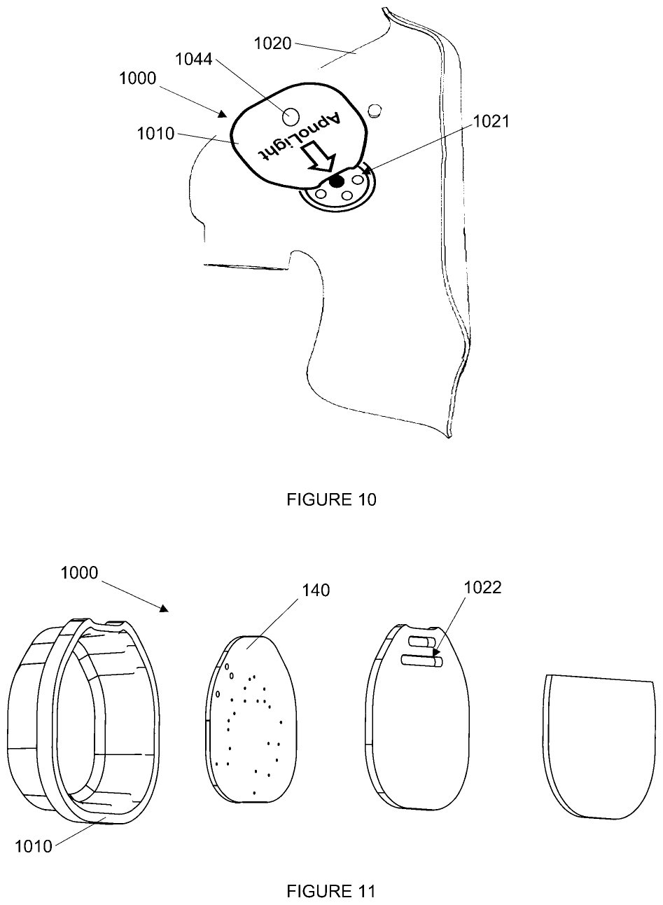

FIGS. 10 and 11 illustrate a breath detection apparatus 1000 according to a preferred embodiment of the present invention for monitoring respiration of a patient. The breath detection apparatus 1000 a housing 1010 and a PCB 140 having a sensing assembly as described above. In some embodiments, the housing 1010 can be omitted.

In the illustrated embodiment, the housing 1010 is adapted to connect to an assistive breathing device in the form of a standard oxygen mask 1020 adjacent a vent in the form of mask ventilation port 1021 of the mask 1020.

The breath detection apparatus 1000 operates substantially similarly to the embodiments described above.

The breath detection apparatus 1000 is intended to provide a low-cost respiratory monitor that can attach to any standard oxygen mask 1020.

The housing 1010 of breath detection apparatus 1000 adheres to the mask 1020 adjacent to the mask ventilation port 1021 and senses the small amount of respiratory gases that exit the port 1021 during exhalation. As can be seen in FIG. 11, a small aperture 1022 is formed in the housing 1020 to allow respiratory gases to enter and exit the housing 1020 via the mask ventilation port 1021.

The housing 1010 does not sit directly in the respiratory airway nor does it require a closed system. The breath detection apparatus 1000 measures the changing humidity and temperature multiple times per second, comparing the most recent values to the previously recorded values.

A rise or fall of the parameters greater than a predefined threshold determines if exhalation or inhalation has occurred.

As the breath detection apparatus 1000 is located outside of the oxygen mask 1020, it senses only a small amount of the exhaled gases and does not sense the effect of inhalation as greatly. Therefore, to clear the sensors of the PCB 140 of hot humid gases between exhalations, the dry cool oxygen flows out of the mask vent holes of the mask ventilation port 1021 and into the housing 1010 enclosure to clear the sensor.

Similar to the embodiments described above, the breath detection apparatus 1000 includes an LED 1044 to visually a breathing state of the patient based on each breath.

Thus, the embodiment of the present invention can be used in essentially an ‘open system’ and does not require the patients exhaled gases to be sealed and directed solely into the housing 1010. The Inventors have found that, using the present embodiment, it is possible hold the breath detection apparatus 1000 near someone's face and accurately detect each individual breath.

Advantageously, the breath detection 1000 does not need to be located in a substantially enclosed space to obtain individual breath indications as the breath detection apparatus 1000 is only concerned with each pair of breaths (comparing the current and previous readings obtained from the sensor). Further advantageously, through the use of short measurement timeframes, the breath detection apparatus 1000 is unencumbered by environmental influences and drift.

Embodiments of the present invention provide an improved way for medical staff to quickly and accurately an absence of patient respiration (i.e. apnoea by detecting the absence of respiration and activating an easily seen light.

Embodiments of the invention provide an alternative method to patient monitoring when CO₂ monitoring is not available or used. The Inventors envision that the detected change in humidity levels can be used as a surrogate to expired CO₂ in monitoring patient respiration.

In a particular advantage of some embodiments, a visual indication of absent respiration is provided when there are no expired flows detected over the humidity sensor. This detection may occur minutes prior to a fall in the patient's oxygenation, thus possibly improving medical staff response time and reducing the risk of hypoxia and permanent injury.

In another advantage of some embodiments, the invention can be retrofitted to existing medical equipment (such as oxygen marks, LMAs and ETTs, for example).

In embodiments of the invention having all of the humidity, pressure and temperature sensors, if one of the sensors fails or readings from the sensors are unreliable (due to excessive humidity, for example) the other 2 sensors can be used to continue to monitor respiration. Thus, redundancy is provided through the use of the humidity, temperature and pressure sensors.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step, etc.

The above detailed description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

Throughout the specification and claims (if present), unless the context requires otherwise, the term “substantially” or “about” will be understood to not be limited to the specific value or range qualified by the terms.

1. A breath detection apparatus for monitoring individual breaths of a patient, the apparatus comprising:
a sensing assembly comprising a humidity sensor, the sensing assembly adapted to be connected to an oxygenation device wherein respiratory gases of a patient are directed over the sensing assembly, the humidity sensor adapted to monitor differences in humidity during a breath of the patient, wherein the humidity sensor generates an electrical signal representing a humidity level in response to measuring the humidity during the breath;
a processor in electrical communication with the sensing assembly to receive electrical signals generated by the humidity sensor and determine a state of the relative humidity during the breath; and
a visual display element controlled by the processor and adapted to display a predetermined visual alert in response to the state of the relative humidity during the breath.

2. The breath detection apparatus of claim 1, wherein the apparatus further comprises a housing adapted to connect to an oxygenation device, the housing having the sensing assembly located therein and adapted to be in fluid communication with the oxygenation device to receive respiratory gases exhaled from the patient to flow over the sensing assembly.

3. The breath detection apparatus of claim 1, wherein the sensing assembly is adapted to monitor differences in humidity during an individual exhalation and/or an inhalation of a respiratory cycle.

4. The breath detection apparatus of claim 1, wherein the sensing assembly comprises only the humidity sensor.

5. The breath detection apparatus of claim 1, wherein the sensing assembly further comprises:
a temperature sensor, wherein the temperature sensor adapted to monitor differences in temperature during the breath, wherein the temperature sensor generates a second electrical signal representing a temperature level in response to measuring the temperature during the breath;
wherein the processor in electrical communication with the sensing assembly receives the first and second electrical signals generated by the humidity sensor and temperature sensor and determines a breathing state during the breath; and
the visual display element is controlled by the processor and adapted to display the predetermined visual alert in response to the breathing state during the breath.

6. The breath detection apparatus of claim 5, wherein the sensing assembly comprises only the humidity sensor and the temperature sensor.

7. A method for breath detection, the method comprising the steps of:
electronically monitoring a humidity level by a humidity sensor connected to an oxygenation device connected to a patient, wherein the humidity sensor receives respiratory gases exhaled by the patient and wherein the humidity sensor generates a first electrical signal representing a humidity value in response to measuring the humidity during a breath;
processing the first electrical signal to determine a state of the relative humidity during the breath; and
generating a predetermined visual alert in response to the state of the relative humidity during the breath.

8. The method of claim 7, wherein the humidity is measured greater than 3 times a second.

9. The method of claim 7, wherein the step of generating a first electrical signal representing the humidity value in response to measure the humidity during a breath further comprises the step of measuring a first humidity level during the breath at a first time; and
measuring a second humidity level during the breath at a second time.

10. The method of the claim 9, herein the second time is after the first time, and wherein the second time is no greater than 500 ms after the first time, or the second time is between 100 ms and 500 ms after the first time, or the second time is between 150 ms and 400 ms after the first time, or the second time is between 200 ms and 300 ms after the first time, or the second time is no greater than 250 ms after the first time, or the second time is 250 ms after the first time.

11. The method of claim 9, wherein the method further comprises the step of comparing the first humidity level with the second humidity level to determine the state of the relative humidity during the breath, wherein comparing the first humidity level with the second humidity level comprises subtracting the first humidity level from the second humidity level.

12. The method of claim 11, wherein the state of the relative humidity during the breath comprises one of the following:
a. a change in the relative humidity during the breath; or
b. no change in the relative humidity during the breath, wherein if a change in the relative humidity during the breath, the state further comprises one of the following:
i. an increase in the relative humidity during the breath; or
ii. a decrease in the relative humidity during the breath; or
iii. no change in the relative humidity during the breath.

13. The method of claim 9, wherein the method further comprises the steps of:
electronically monitoring a temperature level by a temperature sensor, wherein the temperature sensor generates a second electrical signal representing the temperature level in response to measuring the temperature of the breath of the patient;
processing the first and second electrical signals to determine a breathing state during the breath; and
generating the predetermined visual alert in response to the breathing state during the breath.

14. The method of claim 13, wherein the method further comprises the step of measuring:
the first humidity level at the first time;
a first temperature level at the first time;
the second humidity level at the second time; and
a second temperature level at the second time.

15. The method of claim 14, wherein the method further comprises the step of comparing each of the first humidity and temperature levels with the corresponding second humidity and temperature levels to determine the breathing state during the breath, including subtracting the first humidity level from the second humidity level, and subtracting the first temperature level from the second temperature level.

16. The method of claim 15, wherein the step of comparing each of the first humidity and temperature levels with the corresponding second humidity and temperature levels to determine the breathing state during the breath occurs every 250 ms.

17. The breath detection apparatus of claim 5, wherein the breathing state comprises one of the following:
a. a change in each of the humidity and temperature during the breath; or
b. no change in each of the humidity and temperature during the breath, wherein if a change in the relative humidity during the breath, the state further comprises one of the following:
i. an increase in each of the humidity and temperature during the breath; or
ii. a decrease in each of the humidity and temperature during the breath; or
iii. no change in each of the humidity and temperature during the breath.

18. The breath detection apparatus of claim 1, wherein the predetermined visual alert is a unique visual alert based on the determined breathing state during the breath and the apparatus further comprises a wireless communication device for transmitting the predetermined visual alert to a display device and wherein the visual display element comprises an electronically controlled light in electrical communication with the processor, and the processor is programmed to activate the electronically controlled light in response to the breathing state.

19. The method of claim 7, wherein the method further comprises wirelessly transmitting the predetermined visual alert to a display device.

20. A breath detection system for monitoring individual breaths of a patient, the apparatus comprising:
an oxygen mask having a ventilation port;
a housing having an opening and a sensing assembly comprising a humidity sensor therein, the housing positioned over at least a portion of the ventilation port to direct respiratory gases of a patient wearing the oxygen mask into the opening of the housing and over the sensing assembly, the humidity sensor adapted to monitor differences in humidity during a breath of the patient, wherein the humidity sensor generates an electrical signal representing a humidity level in response to measuring the humidity during the breath;
a processor in electrical communication with the sensing assembly to receive electrical signals generated by the humidity sensor and determine a state of the relative humidity during the breath; and
a visual display element controlled by the processor and adapted to display a predetermined visual alert in response to the state of the relative humidity during the breath.
